(19) United States
(12) Patent Application Publication
(10) Pub. No.: US 2010/0282972 A1
(43) Pub. Date: Nov. 11, 2010

(54) INDIRECT RADIATION DETECTOR

(75) Inventors: Raz Carmi, Haifa (IL); Ami Altman, Haifa (IL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/740,392

(22) PCT Filed: Oct. 29, 2008
(86) PCT No.: PCT/IB2008/054455

§ 371 Date: Apr. 29, 2010

(30) Foreign Application Priority Data
Nov. 6, 2007 (CN) 200710185048.4

(51) Int. Cl. G01T 1/166; G01T 1/20; A61B 6/03

(52) U.S. Cl. 250/362; 250/366; 250/363.03; 378/19

(57) ABSTRACT

The present invention relates to an indirect radiation detector for detecting radiation (X), e.g. for medical imaging systems. The detector has an array of pixels (P1-P6), each pixel (P) being sub-divided into at least a first and a second sub-pixel (PE1, PE2). Each sub-pixel has a cross-sectional area (A1, A2) parallel to a surface plane (60) of the array. The cross-sectional area (A1) of the first sub-pixel (PE1) is different, e.g. smaller, from the cross-sectional area (A2) of the second sub-pixel (PE2) to provide a dynamic range of detectable flux densities. Additionally, the first sub-pixel (PE1) has a photosensitive device (PS1) arranged on a side of the sub-pixel, said side being substantially orthogonal to said surface plane of the array of pixels to provide a good optical coupling. The detector allows high-flux photon counting with a relatively simple detector design.

### FIELD OF THE INVENTION

The present invention relates to an indirect radiation detector for detecting radiation, in particular X-ray radiation applied for medical imaging purposes. The invention also relates to a corresponding method of detecting radiation, and a corresponding computer program product.

### BACKGROUND OF THE INVENTION

In typical radiographic imaging systems, e.g. X-ray imaging systems and computed tomography (CT) systems, an X-ray source or emitter radiates X-rays towards an object, e.g. a patient or other objects. The beam traverses through the object, thereby causing an attenuation of the intensity of the X-ray beam. The reduced intensity of the beam can be measured by radiation detectors if appropriately located with respect to the X-ray source and the object being examined.

In other radiographic imaging systems, e.g. positron emission tomography (PET) or single photon emission computed tomography (SPECT), a radiation source is inserted into the object, and an image of the object can be reconstructed by detecting the emitted gamma radiation by means of energy-sensitive photon-counting detectors.

Recently, photon-counting X-ray CT imaging systems have attracted some attention due to their great potential of significantly improving material identification, low-contrast resolution and sensitivity to low radiation doses as compared to a standard CT imaging system (i.e. based on current integration techniques). The photon-counting CT detectors hitherto known are based on direct conversion materials or on fast scintillators which are coupled to optically sensitive devices. Scintillators thereby operate essentially by means of an indirect detection mechanism, which explains why these detectors are also called indirect detectors in the field. Usually, the photon-counting capabilities are used for measuring both the X-ray spectrum and the X-ray photon number in each pixel and in each scan reading. An important aspect is the received X-ray flux density which is the X-ray photon rate per area at the location of the detectors. This quantity can be calculated from the detected photon count number in a given detector element and for a given scan reading. The flux density values (up to a multiplication factor) are essential for the ability to reconstruct an image of the object.

One of the general disadvantages of photon-counting detectors as compared to standard CT detectors which are based on current integration techniques is the relatively low X-ray flux density that can be measured without getting large errors or signal saturation. In a typical clinical CT scan of a human patient, the maximal X-ray flux density at the location of the detectors may be of the order of 10⁹ photons/sec/mm² and even higher. Such a high flux density is mandatory for achieving an overall good performance in terms of short scan time, low image noise and high spatial resolution.

The maximally detectable photon count rate (with tolerable errors) of a given detector pixel is a function of the time constants of the pulse signal in response to an X-ray photon. The time constants define the rise time, the decay time and the width of the pulse. In common detector types, which are appropriate for photon counting X-ray CT, the pulse width is typically of the order of 10 to 50 ns. In some signal-processing techniques optimized for scintillators, the information of the rise pulse alone may be sufficient. The total rise pulse duration may be of the order of 1-5 ns in fast materials. In these ranges of time constants, appropriate fast electronics can be designed so that the rate limitation solely depends on the physical properties of the detector. However, the detection of random photons with temporal Poisson distribution makes it very difficult to reach the required maximal count rates for efficient imaging.

Several known methods can partially mitigate the problem of an insufficiently detectable X-ray flux density in photon-counting CT.

One general approach is to divide the area of the ‘imaging pixel’ (i.e. the effective detector pixel area which is sufficient for proper image reconstruction) into several detector sub-pixels, each of which has an individual signal-processing channel. Within some practical limits, the total achievable flux density is proportional to the number of sub-pixels. After getting the counting results from all sub-pixels, a group of several sub-pixel data can be combined to represent the larger imaging pixel. A clear drawback of this approach is the great increase in the number of individual electronic channels that should be routed and processed. In addition, in some detector types (mainly pixelated scintillators), the structuring of small sub-pixels may introduce technical problems and reduce the effective detection area.

Another known approach is to divide the imaging pixel into several vertical detection layers, one above the other and each having an individual signal-processing channel, cf. US 2006/0056581 (with direct conversion detectors). This technique may also introduce significant complications with respect to photon-counting spectral analysis, because the spectral response of each layer is different than the others. In this case, complicated calibrations and corrections may be required.

Hence, an improved radiation detector that is particularly more efficient and/or reliable would be advantageous.

### OBJECT AND SUMMARY OF THE INVENTION

Accordingly, the invention preferably seeks to mitigate, alleviate or eliminate one or more of the above-mentioned disadvantages singly or in any combination. It is a particular object of the present invention to provide a radiation detector that solves the above-mentioned problems of the prior art with detecting high X-ray flux density in connection with photon counting.

This and several other objects are obtained in a first aspect of the invention by providing an indirect radiation detector for detecting radiation, the detector comprising: an array of pixels, each pixel being sub-divided into at least a first and a second sub-pixel, each sub-pixel having a cross-sectional area parallel to a surface plane of the array of pixels,

wherein the cross-sectional area of the first sub-pixel is different from the cross-sectional area of the second sub-pixel, and wherein the first sub-pixel has a photosensitive device arranged on a side of the sub-pixel, said side being substantially orthogonal to said surface plane of the array of pixels.

The invention is particularly, but not exclusively, advantageous for obtaining an indirect radiation detector that allows high-flux photon counting with a relatively simple detector design. The side-oriented arrangement of a photosensitive device on at least one sub-pixel will typically ensure a good optical coupling between the sub-pixel and the corresponding photosensitive device.

In particular, the present invention may also provide a similar spectral response from the first and the second sub-pixel, which can facilitate easier image reconstruction. Furthermore, the present invention is relatively easy to implement by using existing detector structuring technologies.

In connection with the present invention, it is to be understood that the “surface plane” constitutes a common plane on a boundary of the array of pixels. Due to the large number of pixels required to obtain a sufficient spatial resolution of the radiation detector, the pixels will typically be of a similar or the same size and positioned side by side in the array, rendering the term “surface plane” of the array of pixels reasonably well defined. For an inhomogeneous surface it may be appropriate to define an average surface for the array. The surface plane may be the outer surface of the radiation detector when assembled or it may be a plane situated near such a surface. The impinging radiation will normally be intended to have an incoming direction orthogonal to said surface plane of the array so to give the highest resolution. For some setups though, the radiation may have some deviation from an orthogonal angle of incidence. It is also contemplated that the array of pixels, i.e. the radiation detector may have a certain curvature; the surface plane may accordingly define a tangential plane to the radiation detector at a position of the detector.

In connection with the present invention, it is to be understood that “radiation” may be understood as any kind of electromagnetic radiation carried by a photon having energy in the range of a few electron volts (eV) and higher energies. “Radiation” may thus include ultraviolet (UV), X-ray (soft and hard), and gamma (γ) (soft and hard) radiation. The present invention is particularly advantageous for detecting X-ray radiation in connection with medical imaging.

Advantageously, the second sub-pixel may also have a photosensitive device arranged on a side of the sub-pixel, said side being substantially orthogonal to the surface plane of the array of pixels. Both the first and the second sub-pixel may thus have a side-oriented photosensitive device giving a good optical coupling for both sub-pixels.

Alternatively, the second sub-pixel may have a photosensitive device arranged on a side of the sub-pixel, said side being substantially parallel to the surface plane of the array of pixels. The photosensitive device may thus be on top or at the bottom of the second sub-pixel. Both positions may be easier to manufacture. The side, which is preferably substantially orthogonal to the incoming direction of the radiation, may be positioned on a rear side, i.e. a bottom side of the detector relative to the incoming radiation.

In one embodiment, the first and the second sub-pixel may have different geometrical centers orthogonal to the surface plane of the array of pixels. The pixels can thus be next to each other, making manufacture relatively easy by separating the pixel into smaller elements. In this embodiment, the first and the second sub-pixel may have a substantially rectangular cross-sectional area parallel to a surface plane of the array of pixels. Such box-shaped configurations of the sub-pixels can thus be made conveniently. For the rectangular configuration, the side with the photosensitive device arranged thereon is preferably the side of the first sub-pixel with the largest area so as to ensure maximum optical coupling between the sub-pixel and the corresponding photosensitive device.

In another embodiment, the first and the second sub-pixel may have substantially the same geometrical center orthogonal to the surface plane of the array of pixels, thereby providing a high degree of symmetry that may be beneficial for rebinning, though it may be more difficult to manufacture the detector with this symmetry.

Possibly, a front surface and/or a rear surface of the first sub-pixel is substantially aligned with a front surface and/or a rear surface, respectively, of the second sub-pixel. When the front surfaces are aligned, the surface plane of the array may thus be substantially flat, whereas this need not necessarily be the case in the rear surface alignment configuration.

A ratio between the cross-sectional areas of the first and the second sub-pixel is preferably at least five, or more preferably at least ten. The ratio may also be in the range from 1 to 10, or more preferably 2 to 20 so as to provide a broad range of detectable radiation flux densities.

In an embodiment, each pixel element may be further sub-divided into at least a first, a second and a third sub-pixel, each sub-pixel having a cross-sectional area parallel to a surface plane of the array of pixels. Similarly, the pixel may be sub-divided into four, five, six, seven, eight, nine, ten and a larger number of sub-pixels. With three sub-pixels, the ratio between the cross-sectional areas of the three sub-pixels may range from about 1:5:25 to about 1:10:100. Other ratios may range from about 1:4:8 or about 2:4:8.

In one embodiment, the first and the second sub-pixel may be connected to photon-counting circuitry means so as to apply the invention in connection with high counting rates i.e. higher than 1 Gcps. Specifically, the first and the second sub-pixel may be arranged with the photon-counting circuitry means so as to measure two different sub-ranges of flux density radiation The lowest sub-range is detected by the largest sub-pixel or alternatively by the combination of the two sub-pixels. In the highest sub-range, the photon detection is done only by the sub-pixel with the smallest area. The counted photon numbers in the different sub-pixels can be easily corrected to represent the true radiation flux density which is required for image reconstruction. Correspondingly, three or more sub-pixels may be combined into various detection sub-ranges.

In an embodiment, the photosensitive device may be an avalanche photodiode (APD), a silicon photomultiplier (SiPM), a voltage-biased photodiode, or a photomultiplier tube, or other suitable photosensitive devices capable of converting the light from the sub-pixels into electronically measurable signals.

Typically, the pixels may comprise LSO, LYSO, GSO, YAP, LuAP, or LaBr3, or any alloys thereof for converting the incident radiation into light as is well-known for scintillators.

The present invention also relates to a positron emission tomography (PET) apparatus, a positron single photon emission computed tomography (SPECT) apparatus, a computed tomography (CT) apparatus, or a computed tomography (CT) apparatus with large-area flat-panel imaging comprising a radiation detector according to the first aspect.

In a second aspect, the present invention relates to a method of detecting radiation, the method comprising the steps of:
providing an array of pixels, each pixel being sub-divided into at least a first and a second sub-pixel, each sub-pixel having a cross-sectional area parallel to a surface plane of the array of pixels, and
detecting the radiation by indirect detection,
   wherein the cross-sectional area of the first sub-pixel is different from the cross-sectional area of the second sub-pixel, and
   wherein the first sub-pixel has a photosensitive device arranged on a side of the sub-pixel, said side being substantially orthogonal to said surface plane of the array of pixels.

The first and second aspects of the present invention may each be combined with any one of the other aspects. These and other aspects of the invention are apparent from and will be elucidated with reference to the embodiments described hereinafter.

### BRIEF DESCRIPTION OF THE DRAWINGS

The present invention will now be explained, by way of example only, with reference to the accompanying Figures, in which

FIG. 1 is a schematic representation of a computed tomography (CT) imaging system,

FIG. 2 shows an embodiment of a radiation detector according to the present invention,

FIG. 3 shows another embodiment of a radiation detector according to the present invention,

FIG. 4 shows yet another embodiment of a radiation detector according to the present invention,

FIG. 5 is a top view of two radiation detectors according to the present invention, and

FIG. 6 is a flow chart of a method according to the invention.

### DESCRIPTION OF EMBODIMENTS

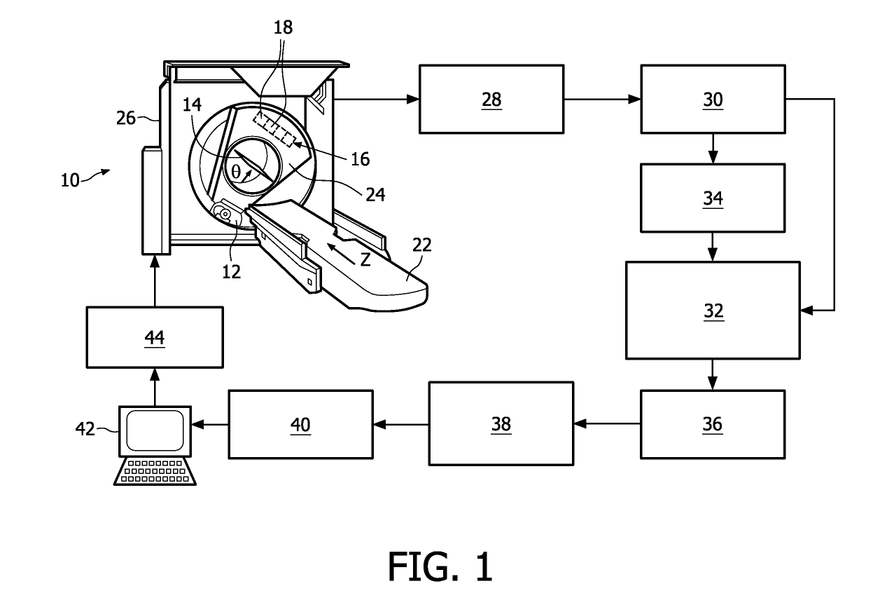

FIG. 1 is a schematic representation of a computed tomography (CT) imaging system, in which a computed tomography scanner 10 houses or supports a radiation source 12, which in one embodiment is an X-ray source, projecting a radiation beam into an examination area 14 defined by the scanner 10. After passing through the examination area 14, the radiation beam is detected by a two-dimensional radiation detector 16 arranged to detect the radiation beam after passing through the examination area 14. The radiation detector 16 includes a plurality of detection modules or detection elements 18. Typically, the X-ray tube produces a diverging X-ray beam having a cone beam, wedge beam, or other beam geometry that expands as it passes through the examination area 14 to substantially fill the area of the radiation detector 16.

An imaging subject is placed on a couch 22 or other support that moves the imaging subject into the examination area 14. The couch 22 is linearly movable along an axial direction designated as Z-direction in FIG. 1. The radiation source 12 and the radiation detector 16 are oppositely mounted with respect to the examination area 14 on a rotating gantry 24, such that rotation of the gantry 24 effects revolving of the radiation source 12 about the examination area 14 so as to provide an angular range of views. The acquired data is referred to as projection data because each detector element detects a signal corresponding to an attenuation line integral taken on a line, narrow cone, or other substantially linear projection extending from the source to the detector element.

During scanning, some portion of the radiation passing along each projection is absorbed by the imaging subject so as to produce a generally spatially varying attenuation of the radiation. The detector elements 18 of the radiation detector 16 sample the radiation intensities across the radiation beam so as to generate radiation absorption projection data. As the gantry 24 rotates in such a way that the radiation source 12 revolves around the examination area 14, a plurality of angular views of projection data is acquired, collectively defining a projection data set that is stored in a buffer memory 28.

For a source-focused acquisition geometry in a multi-slice scanner, readings of the attenuation line integrals or projections of the projection data set stored in the buffer memory 28 can be parameterized as P(γ,β,n), wherein γ is the source angle of the radiation source 12 determined by the position of the rotating gantry 24, β is the angle within the fan (βε[Φ/2, Φ/2], wherein Φ is the fan angle), and n is the detector row number in the Z-direction. A rebinning processor 30 preferably rebins the projection data into a parallel, non-equidistant raster of canonic transaxial coordinates. The rebinning can be expressed as P(γ, β,n) →P(θ,l,n), wherein θ parameterizes the projection number that is composed of parallel readings parameterized by 1 which specifies the distance between a reading and the isocenter, and n is the detector row number in the Z-direction.

The rebinned parallel ray projection data set P(θ,l,n) is stored in a projection data set memory 32. Optionally, the projection data is interpolated by an interpolation processor 34 into equidistant coordinates or into other desired coordinates spacings before storing the projection data P(θ,l,n) in the projection data set memory 32. A reconstruction processor 36 applies filtered back-projection or another image reconstruction technique to reconstruct the projection data set into one or more reconstructed images that are stored in a reconstructed image memory 38. The reconstructed images are processed by a video processor 40 and displayed on a user interface 42 or is otherwise processed or utilized. In one embodiment, the user interface 42 also enables a radiologist, technician, or other operator to interface with a computed tomography scanner controller 44 so as to implement a selected axial, helical, or other computed tomography imaging session.

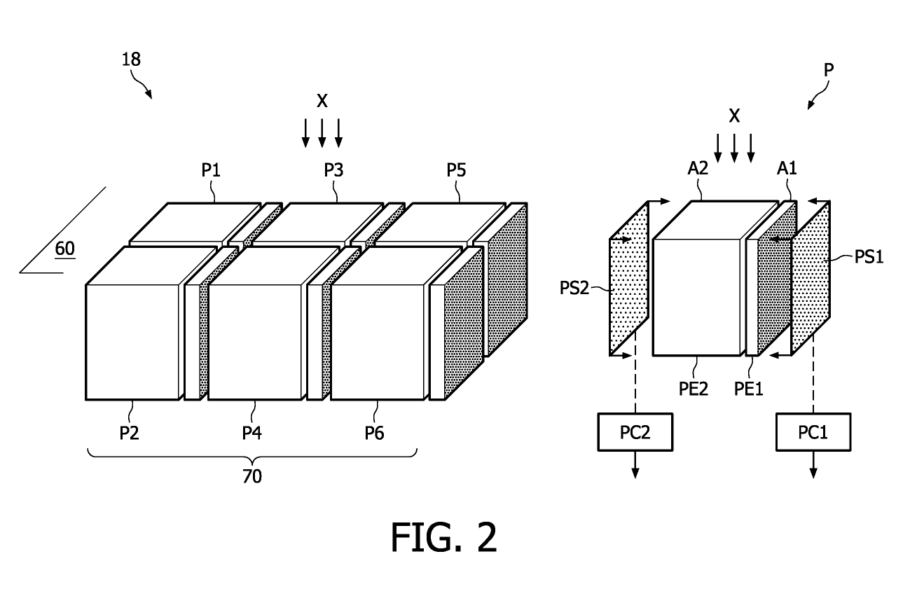

FIG. 2 shows an element 18 of a radiation detector 16 according to the present invention with an array 70 of pixels P1, P2, P3, P4, P5 and P6. The number of pixels may of course typically be much larger for an array, ranging from about a hundred to several ten thousands and even up to several hundred thousands. To obtain a sufficient picture resolution for normal CT purposes, the pixels P1-P6 should have an effective area of the order of 1 mm², though both smaller and larger areas of detection are envisioned with the present invention. The height (i.e. the upwards direction in FIG. 2) of the pixels is typically in the range from 0.5 mm to about 2-3 mm depending on the required stopping power.

The array 70 has an upper surface plane 60 as indicated in the left of FIG. 2. In the displayed configuration of the indirect radiation detector according to the present invention, the radiation X is intended to impinge from above as indicated by three arrows above the array 70.

To the right in FIG. 2, a single pixel P has been separately displayed in an exploded view. The pixel P is sub-divided into a first sub-pixel PE1 and a second sub-pixel PE2, each sub-pixel having a cross-sectional area A1 and A2 parallel to the above-mentioned surface plane 60 of the array 70 of pixels. As can be seen in FIG. 2, the cross-sectional area A1 of the first sub-pixel PE1 is different from the cross-sectional area A2 of the second sub-pixel PE2, i.e. A2 is several times larger than A1; A2>A1. Furthermore, the first and the second sub-pixel PE1, PE2 have photosensitive devices PS1 and PS2, respectively, arranged on the sides. The sides are substantially orthogonal to the surface plane 60 of the array 70 of pixels P1-P6.

The imaging pixel P is thus divided into two non-equal rectangular sub-pixels PE1 and PE2, wherein the two photosensitive devices PS1 and PS2 are coupled from the sides (i.e. substantially parallel to the X-ray radiation X), each one to its corresponding sub-pixel.

In the described configuration, the smaller sub-pixel PE1 has a more efficient optical coupling to the photosensitive device because it is attached through the largest face of the sub-pixel PE1 as compared to a possible situation of attaching PE1 from the bottom side. The technology of attaching and routing photodiodes from the sides of the scintillator pixels is already established and the scintillator configuration can be made by means of known structuring techniques, cf. WO 2006/114716 in the name of the present applicant, which is hereby incorporated by reference in its entirety.

As is usually done after the radiation detector assembly, all faces of the sub-pixels PE1 and PE2 should preferably be covered with optical reflecting material, except those that are attached to the photosensitive devices PS1 and PS2. The sub-pixel with the larger area (or alternatively, the signal sum of the two sub-pixels) gives the counting data in the lower sub-range of X-ray flux density. The sub-pixel with the smaller area alone gives the counting data in the higher sub-range of X-ray flux density.

The surface between PE1 and PE2 may be either parallel to the axial direction or to the angular direction of the imaging system, cf. FIG. 1.

Each of the two photosensitive devices PS1 and PS2 is operably connected with photon-counting signal-processing means PC1 and PC2, as indicated schematically in the lower right portion of FIG. 2.

In the configuration shown in FIG. 2, each sub-pixel has a different geometrical center. Several adaptations should therefore be made in the image reconstruction process. The different sub-pixel coordinates should be considered in the rebinning operation and in the rebinning interpolation steps. In addition, the reconstruction filter prior to back-projection may be adapted as well. In general, if the size of the imaging pixel is designed to allow sufficient spatial sampling after considering the effect of the different sub-pixels, there should be no reconstruction limitations for using these non-equal sub-pixels.

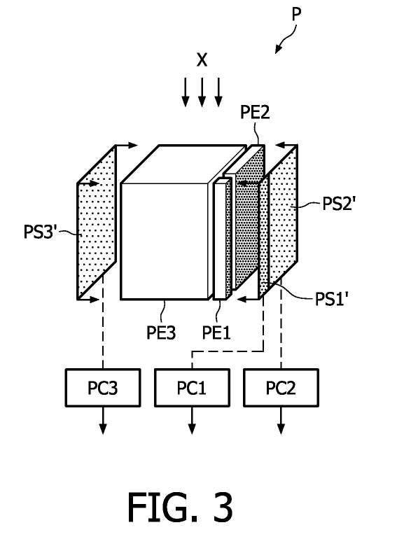

FIG. 3 shows another embodiment of a radiation detector 18 according to the present invention. FIG. 3 describes a configuration similar to that of FIG. 2 but with three non-equal sub-pixels PE1, PE2, and PE3, i.e. three sub-pixels and the three corresponding signal-processing channels PC1, PC2, and PC3, respectively, operably connected to the three photosensitive devices PS1′, PS2′, and PS3′. This configuration can further increase the detectable X-ray flux density due the extra sub-pixel as compared to the embodiment of FIG. 2. However, as the skilled person will recognize, reconstruction adaptation should be implemented in both angular and axial directions.

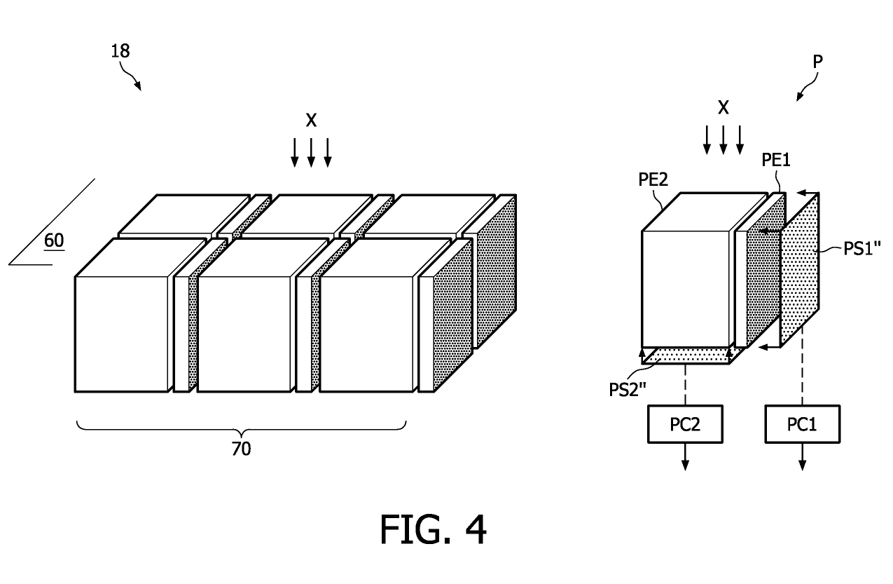

FIG. 4 shows yet another embodiment of a radiation detector 18 according to the present invention. In FIG. 4, the configuration is similar to that of FIG. 2 but in this embodiment the photosensitive device PS2″ of the larger sub-pixel PE2 is attached to the bottom of the scintillator. In this case, the photosensitive devices of many large sub-pixels in the detection array can be made on the same planar chip (along both axial and rotational axes). Another advantage is that there is only a single side-photosensitive chip for each imaging pixel. This allows an increase in the ratio between the active detection area and the non-active area of the detector array.

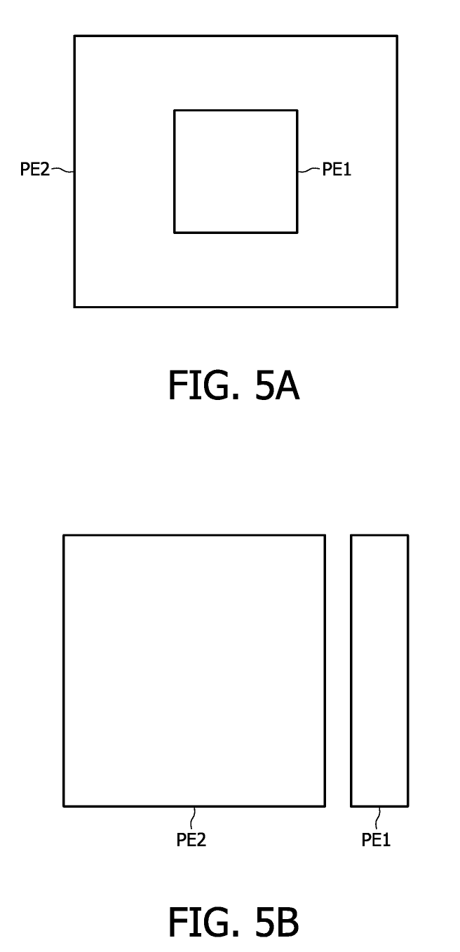

FIG. 5 is a top view of two radiation detectors according to the present invention with X-ray radiation radiated from the front of the paper and into the paper plane as indicated in the Figure.

In part A of FIG. 5, the first and the second sub-pixel PE1 and PE2 have substantially the same geometrical center orthogonal to the surface plane of the array of pixels, i.e. in the paper plane in the view of FIG. 5. The two sub-pixels thus share a common rotational axis which may be beneficial for some rebinning algorithms. In particular, a 180° rotational symmetry with respect to this common axis may be beneficial. It can also be seen that the first and the second sub-pixel PE1 and PE2 have the same aspect ratio, i.e. ratio between height and width as seen in the view of FIG. 5. The first and the second sub-pixel PE1 and PE2 can, however, have a different aspect ratio and still have a common geometrical center orthogonal to the surface plane of the array of pixels, i.e. in the paper plane in the view of FIG. 5.

In part B of FIG. 5, the first and the second sub-pixel PE1 and PE2 have different geometrical centers orthogonal to the surface plane of the array of pixels, i.e. the paper plane in the view of FIG. 5. This is similar to the configurations shown in FIGS. 2, 3 and 4, as described above in more detail.

As shown, the first and the second sub-pixel PE1 and PE2 have a rectangular cross-sectional area parallel to a surface plane of the array of pixels i.e. in the paper plane in the view of FIG. 5.

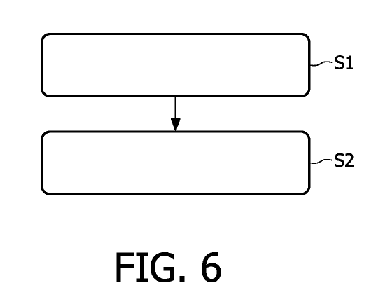

FIG. 6 is a flow chart of a method according to the invention. The method comprises the following steps.

Step S1 providing an array of pixels P1-P6, each pixel P being sub-divided into at least a first and a second sub-pixel PE1, PE2, each sub-pixel having a cross-sectional area A1 and A2 parallel to a surface plane 60 of the array of pixels, and

Step S2 detecting the radiation X by indirect detection,

wherein the cross-sectional area A1 of the first sub-pixel PE1 is different from the cross-sectional area A2 of the second sub-pixel PE2, and
wherein the first sub-pixel PE1 has a photosensitive device PS1 arranged on a side of the sub-pixel, said side being substantially orthogonal to said surface plane of the array of pixels.

The invention can be implemented in any suitable form including hardware, software, firmware or any combination of these. The invention, or some of its features, can be implemented as computer software running on one or more data processors and/or digital signal processors. The elements and components of an embodiment of the invention may be physically, functionally and logically implemented in any suitable way. Indeed, the functionality may be implemented in a single unit, in a plurality of units or as part of other functional units. As such, the invention may be implemented in a single unit, or may be physically and functionally distributed between different units and processors.

Although the present invention has been described in connection with the specified embodiments, it is not intended to be limited to the specific form set forth herein. The scope of the present invention is limited only by the appendant claims. In the claims, use of the verb “comprise” and its conjugations does not exclude the presence of other elements or steps. Additionally, although individual features may be included in different claims, these may possibly be advantageously combined, and the inclusion in different claims does not imply that a combination of features is not feasible and/or advantageous. In addition, singular references do not exclude a plurality. Thus, references to “a”, “an”, “first”, “second” etc. do not preclude a plurality. Furthermore, reference signs in the claims shall not be construed as limiting their scope.

1. An indirect radiation detector for detecting radiation (X), the detector comprising:
an array of pixels (P1-P6), each pixel (P) being sub-divided into at least a first and a second sub-pixel (PE1, PE2), each sub-pixel having a cross-sectional area (A1, A2) parallel to a surface plane (60) of the array of pixels,
wherein the cross-sectional area (A1) of the first sub-pixel (PEI) is different from the cross-sectional area (A2) of the second sub-pixel (PE2), and wherein the first sub-pixel (PEI) comprises a photosensitive device (PSI) arranged on a side of the sub-pixel, said side being substantially orthogonal to said surface plane of the array of pixels.

2. The detector according to claim 1, wherein the second sub-pixel (PE2) comprises a photosensitive device (PS2) arranged on a side of the sub-pixel (PE2), said side being substantially orthogonal to the surface plane of the array of pixels.

3. The detector according to claim 1, wherein the second sub-pixel (PE2) comprises a photosensitive device (PS2) arranged on a side of the sub-pixel (PE2), said side being substantially parallel to the surface plane of the array of pixels.

4. The detector according to claim 3, wherein the side substantially orthogonal to the incoming direction (U) of the radiation (X) is positioned on a rear side of the detector relative to the incoming radiation (X).

5. The detector according to claim 1, wherein the first and the second sub-pixel (PE1, PE2) have different geometrical centers orthogonal to the surface plane of the array of pixels.

6. The detector according to claim 5, wherein the first and the second sub-pixel (PEI, PE2) have a substantially rectangular cross-sectional area (AI, A2) parallel to a surface plane of the array of pixels.

7. The detector according to claim 1, wherein the first and the second sub-pixel (PEI, PE2) have substantially the same geometrical center orthogonal to the surface plane of the array of pixels.

8. The detector according to claim 1, wherein a front surface and/or a rear surface of the first sub-pixel (PEI) is substantially aligned with a front surface and/or a rear surface, respectively, of the second sub-pixel (PE2).

9. The detector according to claim 6, wherein the side with the photosensitive device (PSI) arranged thereon is the side of the first sub-pixel (PEI) with the largest area.

10. The detector according to claim 1, wherein a ratio between the cross-sectional areas (A2, AI) of the second and the first sub-pixel (PE2, PEI) is at least five, preferably at least ten.

11. The detector according to claim 1, wherein each pixel (P) is sub-divided into at least a first, a second and a third sub-pixel (PEI, PE2, PE3), each sub-pixel having a cross-sectional area (AI, A2, A3) parallel to a surface plane (60) of the array of pixels.

12. The detector according to claim 11, wherein the ratio between the cross-sectional areas (AI, A2, A3) of the three sub-pixels (PEI, PE2, PE3) is in the range from about 1:5:25 to about 1:10:100.

13. The detector according to claim 1, wherein the first and the second sub-pixel (PEI, PE2) are coupled to photon-counting circuitry means.

14. The detector according to claim 13, wherein the first and the second sub-pixel are arranged with the photon-counting circuitry means to measure two different sub-ranges of flux density radiation.

15. The detector according to claim 1, wherein the photosensitive (PS) device is an avalanche photodiode (APD), a silicon photomultiplier (SiPM), a voltage-biased photodiode, or a photomultiplier tube.

16. The detector according to claim 1, wherein the pixels comprise LSO, LYSO, GSO, YAP, LuAP, or LaBr3, or any alloys thereof.

17. A positron emission tomography (PET) apparatus comprising a radiation detector, wherein the radiation detector comprises:
an array of pixels (P1-P6), each pixel (P) being sub-divided into at least a first and a second sub-pixel (PEI, PE2), each sub-pixel having a cross-sectional area (AI, A2) parallel to a surface plane (60) of the array of pixels,
wherein the cross-sectional area (AI) of the first sub-pixel (PEI) is different from the cross-sectional area (A2) of the second sub-pixel (PE2), and
wherein the first sub-pixel (PEI) comprises a photosensitive device (PSI) arranged on a side of the sub-pixel, said side being substantially orthogonal to said surface plane of the array of pixels.

18. A positron single photon emission computed tomography (SPECT) apparatus comprising a radiation detector, wherein the radiation detector comprises:
an array of pixels (P1-P6), each pixel (P) being sub-divided into at least a first and a second sub-pixel (PEI, PE2), each sub-pixel having a cross-sectional area (AI, A2) parallel to a surface plane (60) of the array of pixels,
wherein the cross-sectional area (AI) of the first sub-pixel (PEI) is different from the cross-sectional area (A2) of the second sub-pixel (PE2), and
wherein the first sub-pixel (PEI) comprises a photosensitive device (PSI) arranged on a side of the sub-pixel, said side being substantially orthogonal to said surface plane of the array of pixels.

19. A computed tomography (CT) apparatus comprising a radiation detector, wherein the radiation detector comprises:
an array of pixels (P1-P6), each pixel (P) being sub-divided into at least a first and a second sub-pixel (PEI, PE2), each sub-pixel having a cross-sectional area (AI, A2) parallel to a surface plane (60) of the array of pixels,
wherein the cross-sectional area (AI) of the first sub-pixel (PEI) is different from the cross-sectional area (A2) of the second sub-pixel (PE2), and wherein the first sub-pixel (PEI) comprises a photosensitive device (PSI) arranged on a side of the sub-pixel, said side being substantially orthogonal to said surface plane of the array of pixels.

20. A computed tomography (CT) apparatus with large-area flat-panel imaging comprising a radiation detector, wherein the radiation detector comprises:
an array of pixels (P1-P6), each pixel (P) being sub-divided into at least a first and a second sub-pixel (PEI, PE2), each sub-pixel having a cross-sectional area (AI, A2) parallel to a surface plane (60) of the array of pixels,
wherein the cross-sectional area (AI) of the first sub-pixel (PEI) is different from the cross-sectional area (A2) of the second sub-pixel (PE2), and wherein the first sub-pixel (PEI) comprises a photosensitive device (PSI) arranged on a side of the sub-pixel, said side being substantially orthogonal to said surface plane of the array of pixels.

21. A method of detecting radiation (X), the method comprising the steps of:
providing an array of pixels (P1-P6), each pixel (P) being sub-divided into at least a first and a second sub-pixel (PEI, PE2), each sub-pixel having a cross-sectional area (AI, A2) parallel to a surface plane (60) of the array of pixels, and
detecting the radiation (X) by indirect detection, wherein the cross-sectional area (AI) of the first sub-pixel (PEI) is different from the cross-sectional area (A2) of the second sub-pixel (PE2), and wherein the first sub-pixel (PEI) comprises a photosensitive device (PSI) arranged on a side of the sub-pixel, said side being substantially orthogonal to said surface plane of the array of pixels.
